SEQUENCE LISTING (1 sequence)

SEQ ID NO: 1 - DNA, 4409 bp, zea maize
ggatccaccatgggtaattccctcggcaacaacctcctctaccagggcttcctcacctccaccgtgctcgagcacaagtcccgcatctccccgccgtgcgtgggcggcaactccctcttccagcagcaggtgatctccaagtccccgctctccaccgagttccgcggcaaccgcctcaaggtgcagaagaagaagatcccgatggagaagaagcgcgccttctcctcctccccgcacgccgtgctcaccaccgacacctcctccgaactcgccgagaagttctccctcggcggcaacatcgaactccaggtggacgtgcgcccgccgacctccggcgacgtgtccttcgtggacttccaggtgaccaacggctccgacaagctcttcctccactggggcgccgtgaagttcggcaaggagacctggtccctcccgaacgaccgcccggacggcaccaaggtgtacaagaacaaggccctccgcaccccgttcgtgaagtccggctccaactccatcctccgcctcgagatccgcgacaccgccatcgaggccatcgagttcctcatctacgacgaggcccacgacaagtggatcaagaacaacggcggcaacttccgcgtgaagctctcccgcaaggagatccgcggcccggacgtgtccgtgccggaggaactcgtgcagatccagtcctacctccgctgggagcgcaagggcaagcagaactacccgccggagaaggagaaggaggagtacgaggctgctcgcaccgtgctccaggaggagatcgctcgcggtgcctccatccaggacatccgcgcccgcctcaccaagaccaacgacaagtcccagtccaaggaggagccgctccacgtgaccaagtccgacatcccggacgacctcgcccaggcccaggcctacatccgctgggagaaggccggcaagccgaactacccgccggagaagcagatcgaggaactcgaggaggcccgccgcgaactccagctcgaactcgagaagggcatcaccctcgacgaactccgcaagaccatcaccaagggcgagatcaagaccaaggtggagaagcacctcaagcgctcctccttcgccgtggagcgcatccagcgcaagaagcgcgacttcggccacctcatcaacaagtacacctcctcccctgccgtgcaggtgcagaaggtgctcgaggagccaccagccctctccaagatcaagctctacgccaaggagaaggaggagcagatcgacgacccgatcctcaacaagaagatcttcaaggtggacgacggcgaactcctcgtgctcgtggccaagtcctccggcaagaccaaggtgcacctcgccaccgacctcaaccagccgatcaccctccactgggccctctccaagtccccgggcgagtggatggtgccgccgtcctccatcctcccgccgggctccatcatcctcgacaaggccgccgagaccccgttctccgcctcctcctccgacggcctcacctccaaggtgcagtccctcgacatcgtgatcgaggacggcaacttcgtgggcatgccgttcgtgctcctctccggcgagaagtggatcaagaaccagggctccgacttctacgtgggcttctccgccgcctccaagctcgccctcaaggctgctggcgacggctccggcaccgccaagtccctcctcgacaagatcgccgacatggagtccgaggcccagaagtccttcatgcaccgcttcaacatcgccgccgacctcatcgaggacgccacctccgccggcgaactcggcttcgccggcatcctcgtgtggatgcgcttcatggccacccgccagctcatctggaacaagaactacaacgtgaagccgcgcgagatctccaaggcccaggaccgcctcaccgacctcctccagaacgccttcacctcccacccgcagtaccgcgagatcctccgcatgatcatgtccaccgtgggtcgcggtggcgagggcgacgtgggccagcgcatccgcgacgagatcctcgtgatccagcgcaacaacgactgcaagggcggcatgatgcaggagtggcaccagaagctccacaacaacacctccccggacgacgtggtgatctgccaggccctcatcgactacatcaagtccgacttcgacctcggcgtgtactggaagaccctcaacgagaacggcatcaccaaggagcgcctcctctcctacgaccgcgccatccactccgagccgaacttccgcggcgaccagaagggcggcctcctccgcgacctcggccactacatgcgcaccctcaaggccgtgcactccggcgccgacctcgagtccgccatcgccaactgcatgggctacaagaccgagggcgagggcttcatggtgggcgtgcagatcaacccggtgtccggcctcccgtccggcttccaggacctcctccacttcgtgctcgaccacgtggaggacaagaacgtggagaccctcctcgagcgcctcctcgaggcccgcgaggaactccgcccgctcctcctcaagccgaacaaccgcctcaaggacctcctcttcctcgacatcgccctcgactccaccgtgcgcaccgccgtggagcgcggctacgaggaactcaacaacgccaacccggagaagatcatgtacttcatctccctcgtgctcgagaacctcgccctctccgtggacgacaacgaggacctcgtgtactgcctcaagggctggaaccaggccctctccatgtccaacggcggcgacaaccactgggccctcttcgccaaggccgtgctcgaccgcacccgcctcgccctcgcctccaaggccgagtggtatcaccacctcctccagccgtccgccgagtacctcggctccatcctcggcgtggaccagtgggccctcaacatcttcaccgaggagatcatccgcgccggctccgccgcctccctctcctccctcctcaaccgcctcgacccggtgctccgcaagaccgccaacctcggctcctggcagatcatctccccggtggaggccgtgggctacgtggtggtggtggacgaactcctctccgtgcagaacgagatctacgagaagccgaccatcctcgtggccaagtccgtgaagggcgaggaggagatcccggacggcgccgtggccctcatcaccccggacatgccggacgtgctctcccacgtgtccgtgcgcgcccgcaacggcaaggtgtgcttcgccacctgcttcgacccgaacatcctcgccgacctccaggccaaggagggccgcatcctcctcctcaagccgaccccgtccgacatcatctactccgaggtgaacgagatcgaactccagtcctcctccaacctcgtggaggccgagacctccgccaccctccgcctcgtgaagaagcagttcggcggctgctacgccatctccgccgacgagttcacctccgagatggtgggcgccaagtcccgcaacatcgcctacctcaagggcaaggtgccgtcctccgtgggcatcccgacctccgtggccctcccgttcggcgtgttcgagaaggtgctctccgacgacatcaaccagggcgtggccaaggaactccagatcctcatgaagaagctctccgagggcgacttctccgccctcggcgagatccgcaccaccgtgctcgacctctccgccccggcccagctcgtgaaggaactcaaggagaagatgcagggctccggcatgccgtggccgggcgacgagggcccgaagcgctgggagcaggcctggatggccatcaagaaggtgtgggcctccaagtggaacgagcgcgcctacttctccacccgcaaggtgaagctcgaccacgactacctctgcatggccgtgctcgtgcaggagatcatcaacgccgactacgccttcgtgatccacaccaccaacccgtcctccggcgacgactccgagatctacgccgaggtggtgcgcggcctcggcgagaccctcgtgggagcctacccaggacgcgcactctccttcatctgcaagaagaaggacctcaactccccgcaggtgctcggctacccgtccaagccgatcggcctcttcatcaagcgctccatcatcttccgctccgactccaacggcgaggacctcgagggctacgccggcgccggcctctacgactccgtgccgatggacgaggaggagaaggtggtgatcgactactcctccgacccgctcatcaccgacggcaacttccgccagaccatcctctccaacatcgcccgcgccggccacgccatcgaggaactctacggctccccgcaggacatcgagggcgtggtgcgcgacggcaagatctacgtggtgcagacccgcccgcagatgtagagctc

(19) United States
(12) Patent Application Publication
(10) Pub. No.: US 2006/0282917 A1
(43) Pub. Date: Dec. 14, 2006

(54) Modified starch, uses, methods for production thereof

(75) Inventors: Michael B. Lanahan, Research Triangle Park, NC (US); Shib S. Basu, Apex, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 10/556,276

(22) PCT Filed: May 24, 2004
(86) PCT No.: PCT/US04/16291

§ 371 Date: Nov. 15, 2005

Related U.S. Application Data
(60) Provisional application No. 60/472,533, filed on May 22, 2003.

(51) Int. Cl. A01H 1/00; C12P 19/04; C08B 31/00; C12N 15/82; C12N 5/04; A23K 1/00

(52) U.S. Cl. 800/284; 435/101; 435/412; 435/468; 536/102; 426/635

(57) ABSTRACT

The present invention relates to modified starch, as well as production and uses thereof. The starch has modified properties of viscosity and a modified phosphate content. The present invention also relates to a nucleic acid molecule encoding a codon-optimized form of a maize R1 protein set forth in SEQ ID NO:1.

### SUMMARY OF THE INVENTION

The present invention relates to modified starch, as well as production and uses thereof. The starch has modified properties of viscosity and a modified phosphate content.

### BRIEF DESCRIPTION OF THE FIGURES

FIG. 1 depicts an Agrobacterium-vector containing a PCR-amplified potato-R1 as insert.

FIG. 2 depicts an Agrobacterium-vector with synthetic R1 as insert.

FIG. 3 is a graph showing estimations of glucose 6-phosphate after complete hydrolysis of starch and Increased phosphorylation of R1-cornstarch.

FIG. 4 shows the relative swelling-power of R1-cornstarch compared to non-transgenic cornstarch.

FIG. 5 shows the relative solubility of the R1-cornstarch compared to the non-transgenic cornstarch.

FIG. 6 shows an HPLC analysis demonstrating in vitro digestibility of R1-corn flour under simulated digestive conditions.

FIG. 7 shows the susceptibility of R1-corn flour to enzymatic hydrolysis by starch hydrolyzing enzymes.

FIG. 8 shows the effect of incubation time and enzyme concentration on the rate of hydrolysis of R1-cornstarch.

FIG. 9 demonstrates the fermentability of R1 cornstarch.

FIG. 10 shows the starch phosphorylation level of T1 seed expressing synthetic R1 (codon-optimized).

### DETAILED DESCRIPTION OF THE INVENTION

The protein encoded by nucleic acid molecules described herein is an R1 protein which influences starch synthesis and/or modification. It was found that changes in the amount of the protein in plant cells lead to changes in the starch metabolism of the plant, and in particular to the synthesis of starch with modified physical and chemical properties.

Using the nucleic acid molecules encoding R1 protein allowed production of transgenic plants, by means of recombinant DNA techniques, synthesizing a modified starch that differs from the starch synthesized in wild-type plants with respect to its structure and its physical and chemical properties. To achieve this, the nucleic acid molecules encoding R1 protein were linked to regulatory elements, which ensure transcription and translation in plant cells, and were then introduced into plant cells. The nucleic acid molecule of the invention is preferably a maize optimized nucleic acid sequence, such as the sequence set forth in SEQ ID NO:1.

Therefore, the present invention uses transgenic plant cells containing a nucleic acid molecule encoding R1 protein whereby the nucleic acid molecule is linked to regulatory elements that ensure the transcription in plant cells. The regulatory elements are preferably heterologous with respect to the nucleic acid molecule.

Employing methods known to the skilled artisan, the transgenic plant cells may be regenerated to whole plants. A further subject matter of the invention includes plants that contain the above-described transgenic plant cells. The transgenic plants may in principle be plants of any desired species, i.e. they may be monocotyledonous as well as dicotyledonous plants. Preferably, the plant and plant cells utilized in the invention are transgenic maize or transgenic rice.

Due to the expression or the additional expression of a nucleic acid molecule encoding R1 protein, the transgenic plant cells and plants used in the invention synthesize a starch which is modified when compared to starch from wild-type plants, i.e. non-transformed plants, particularly with respect to the viscosity of aqueous solutions of this starch and/or to the phosphate content

Hence, the starch obtainable from the transgenic plant cells and plants of the invention is the subject matter of the present invention.

Covalent derivatization of starch with ionic functional group(s) increases its solubility and swelling capacity in any ionic medium, making the modified starch molecules more accessible to other molecules (e.g., modifying agents chemicals and/or enzymes). For example, covalently modifying glucose residues of starch with an ionic phosphate group can increase the affinity of the starch molecules for water or any polar solvent. This derivatization can also assists the swelling of the starch through electrical repulsion between the doubly negatively charged phosphate groups attached to strands of glucose residues. The swelled and hydrated phosphorylated starch is more susceptible to attack by amodifying agent, including for example, hydrolytic enzyme, chemicals and/or enzymes for further derivatization.

Examples of modifying agents include, but are not limited to, cross linking agents such as phosphorus oxychloride, sodium trimetaphosphate, adipic-acetic anhydride etc. and substituting agents like proplene oxide, 1-octenyl succinic anhydride, and acetic anhydride.

The starch obtainable from the transgenic plants of the invention may be used for food and feed applications. The use of the starch, derivatized with ionic functional group(s) (e.g. phosphate) may not only increase the proportion of starch available for hydrolysis, but may also increase the rate of starch hydrolysis and/or decrease the enzyme requirement to achieve complete hydrolysis.

The modified starch of the invention may be used, for example, in the following:

In animal feed. Formulation of diet with easily digestible starch and hence more extractable dietary energy. While the modified starch may be used in the diets of any animal, it is preferred that such starch is used in the diets of monogastric animals, including, but not limited to, chicken and pig. The modified starch is also useful in diets for ruminants, such as cows, goats, and sheep.

In human food. Formulation of diet with easily digestible starch and hence more extractable dietary energy.

In the fermentation process, as fermentable raw-material. Starch, usefulin different fermentation processes (e.g. ethanol production), is first broken down to easily fermentable sugars (degree of polymerization usually less than or equal to 3) by amylase and/or glucoamylase. This enzymatic hydrolysis is followed by fermentation, which converts sugars to various fermentation products (e.g. ethanol). Hence, a starch that can be more easily (in less time and/or by using of lower enzyme dose) hydrolyzed by amylase and/or glucoamylase may serve as a better starting substrate for the fermentation process.

The modified starch of the invention may be used in any fermentation process, including, but not limited to, ethanol production, lactic acid production, and polyol production (such as glyercol production).

Improved digestibility of the modified starch of the invention, i.e., the R1-cornstarch, at ambient temperature can make the ‘raw-starch fermentation’ process economically profitable by making larger portion of the starch available and accessible for hydrolysis by the hydrolases.

Accordingly, the modified starch of the invention may be used in raw starch fermentation. In the raw starch fermentation, the starch is not liquefied before enzymatic hydrolysis, the hydrolysis is carried at ambient temperature simultaneously with the fermentation process.

Derivatization of starch in-planta using the method of the invention, namely, the method of transgenic expression of R1-protein (a glucan dikinase) allows improved starch solubility and swelling power and increased starch digestibility when used as feed, food or as a fermentable substrate.

Also included in the invention is a method to prepare a solution of hydrolyzed starch product comprising treating a plant or plant part comprising starch granules under conditions which activate the R1 polypeptide thereby processing the starch granules to form an aqueous solution comprising hydrolyzed starch product. The plant or plant part utilized in the invention is a transgenic plant or plant part, the genome of which is augmented with an expression cassette encoding an R1 polypeptide. The hydrolyzed starch product may comprise a dextrin, maltooligosaccharide, glucose and/or mixtures thereof. The method may further comprise isolating the hydrolyzed starch product and/or fermenting the hydrolyzed starch product

The R1 polypeptide is preferably expressed in the endosperm. The sequence of the R1 gene may be operably linked to a promoter and to a signal sequence that targets the enzyme to the starch granule.

The invention also encompasses a method of preparing hydrolyzed starch product comprising treating a plant or plant part comprising starch granules under conditions which activate the R1 polypeptide thereby processing the starch granules to form an aqueous solution comprising a hydrolyzed starch product The plant or plant part utilized in the invention is a transgenic plant or plant part, the genome of which is augmented with an expression cassette encoding an R1 polypeptide.

Also included is a method of preparing fermentation products, such as ethanol, comprising treating a plant or plant part comprising starch granules under conditions to activate the R1 polypeptide thereby digesting polysaccharide to form oligosaccharide or fermentable sugar, and incubating the fermentable sugar under conditions that promote the conversion of the fermentable sugar or oligosaccharide into ethanol. The plant or plant part utilized in the invention is a transgenic plant or plant part, the genome of which is augmented with an expression cassette encoding an R1 polypeptide.

The plant part may be a grain, fruit, seed, stalks, wood, vegetable or root. Preferably the plant part is obtained from a plant such as oats, barley, corn or rice. Fermentation products include, but are not limited to, ethanol, acetic acid, glycerol, and lactic acid.

Also encompassed is a method of preparing maltodextrin comprising mixing transgenic grain with water, heating said mixture, separating solid from the dextrin syrup generated, and collecting the maltodextrin. In addition, a method of preparing dextrins or sugars from grain expressing R1 is included.

The invention is further directed to a method of producing fermentable sugar employing transgenic grain expressing R1.

The increased solubility and swelling power of the modified starches derivatized with ionic functional groups make them more susceptible to attack not only by hydrolytic enzymes but also by any modifying agent. Hence the modified starches may be even further modified by additional enzymatic and/or chemical modifications. Swelled and solvated starch may allow increased penetration of the modifying agent into the starch molecule/granule, and therefore may accommodate a higher degree of substitution, as well as uniform distribution of the functional groups in the starch molecule/granule.

The invention will be further described by the following methods and examples, which are not intended to limit the scope of the invention in any manner.

### EXAMPLES

### Example 1

Constructs for Expression of R1 in Corn.

PCR Amplification and Cloning of Potato R1-cDNA.

The full-length cDNA was amplified by PCR from a cDNA-library of potato (Solanum tuberosum) tissues using primers R1-5′-pr: 5′- T GCA GCC ATG GGT AAT TCC TTA GGG AAT AAC-3′ and R1-3′-pr: 5′- TC CAA GTC GAC TCA CAT CTG AGG TCT TGT CTG -3′ designed from GenBank Accession No. Y09533 [Lorberth R., Ritte G., Willmitzer L., Kossmann J., Nature Biotech. 1998, 16, 473-477]. The amplified DNA was cloned into pCR vector using TA cloning kit (Invitrogen). The sequence of the insert was confirmed and then moved (cut and ligated) into agro-transformation vector described below.

Construction of Maize Codon-Optimized Genes for R1:

The amino acid sequence for R1-protein from was obtained from the literature [Lorberth R., Ritte G., Willmitzer L., Kossmann J., Nature Biotech. 1998, 16, 473-477]. Based on the published amino acid sequence of the protein, the maize-optimized synthetic gene (SEQ ID NO:1) encoding the R1 was designed.

Isolation of Promoter Fragments (γ-zein) for Endosperm-Specific Expression

The (γ-zein) promoter used in the constructs described herein was isolated as disclosed in International Publication No. WO 03/018766, published Mar. 6, 2003, which is incorporated by reference in its entirety herein.

Construction of Agro-Transformation Vectors for R1:

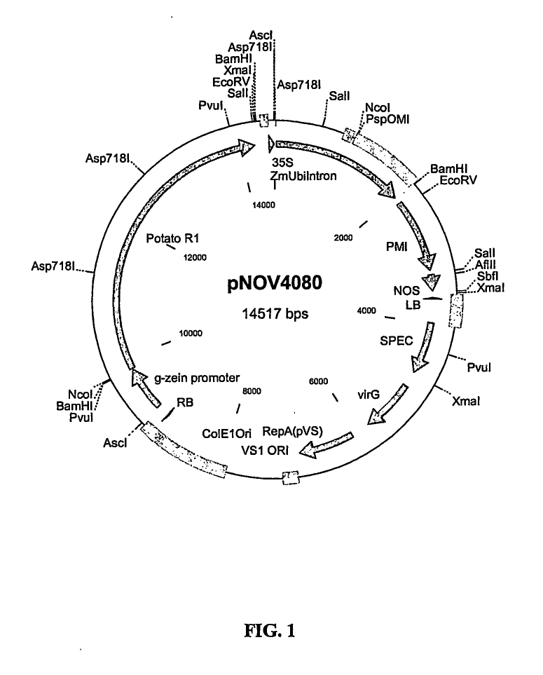

The plasmid pNOV4080 (FIG. 1) was constructed by ligating the PCR amplified potato R1-DNA (NcoI and SalI are the two flanking restriction sites) behind (i.e., 3′ of) the maize γ-zein promoter. The transformation into maize was carried out via Agrobacterium infection. The transformation vector contained the phosphomannose isomerase (PMI) gene that allows selection of transgenic cells with mannose. Transformed maize plants were either self-pollinated and seed was collected for analysis.

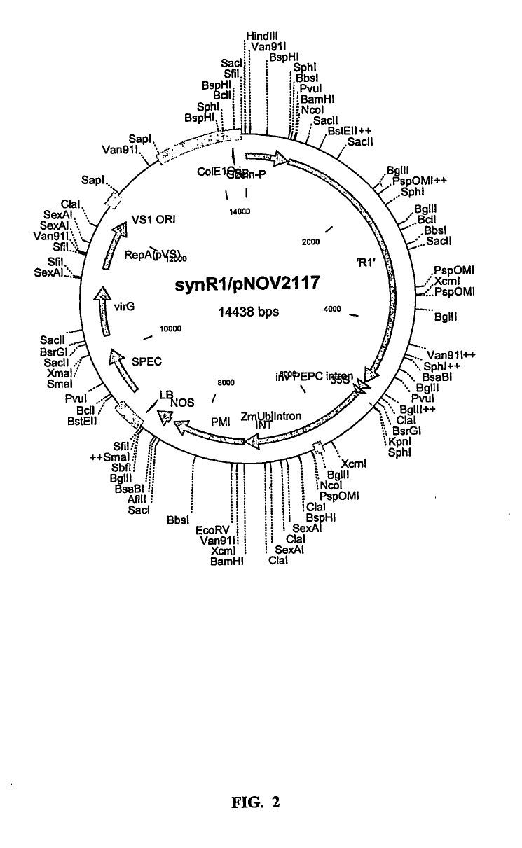

The plasmid pNOV 2117 (FIG. 2) was constructed in a similar manner. The insert is a synthetically made R1-DNA with maize-codon optimized sequence coding for the amino acid sequence shown in SEQ ID NO:1. A description of pNOV2117 is disclosed in International Publication No. WO 03/018766, published Mar. 6, 2003.

### Example 2

Agrobacterium Transformation.

A. Transformation Plasmids and Selectable Marker.

The genes used for transformation were cloned into a vector suitable for maize transformation. Vectors used in this example contained the phosphomannose isomerase (PMI) gene for selection of transgenic lines (Negrotto et al. (2000) Plant Cell Reports 19: 798-803).

B. Preparation of Agrobacterium tumefaciens.

Agrobacterium strain LBA4404 (pSB1) containing the plant transformation plasmid was grown on YEP (yeast extract (5 g/L), peptone (10 g/L), NaCl (5 g/L), 15 g/l agar, pH 6.8) solid medium for 2-4 days at 28° C. Approximately 0.8×10⁹ Agrobacterium were suspended in LS-inf media supplemented with 100 μM As (Negrotto et al., (2000) Plant Cell Rep 19: 798-803). Bacteria were pre-induced in this medium for 30-60 minutes.

C. Inoculation.

Immature embryos from A188 or other suitable genotype were excised from 8-12 day old ears into liquid LS-inf+100 μM As. Embryos were rinsed once with fresh infection medium. Agrobacterium solution was then added and embryos were vortexed for 30 seconds and allowed to settle with the bacteria for 5 minutes. The embryos were then transferred scutellum side up to LSAs medium and cultured in the dark for two to three days. Subsequently, between 20 and 25 embryos per petri plate were transferred to LSDc medium supplemented with cefotaxime (250 mg/l) and silver nitrate (1.6 mg/l) and cultured in the dark for 28° C. for 10 days.

D. Selection of Transformed Cells and Regeneration of Transformed Plants.

Immature embryos producing embryogenic callus were transferred to LSD1M0.5S medium. The cultures were selected on this medium for 6 weeks with a subculture step at 3 weeks. Surviving calli were transferred to Reg1 medium supplemented with mannose. Following culturing in the light (16 hour light/8 hour dark regiment), green tissues were then transferred to Reg2 medium without growth regulators and incubated for 1-2 weeks. Plantlets are transferred to Magenta GA-7 boxes (Magenta Corp, Chicago Ill.) containing Reg3 medium and grown in the light. After 2-3 weeks, plants were tested for the presence of the PMI genes and other genes of interest by PCR. Positive plants from the PCR assay were transferred to the greenhouse.

Expression of R1 in Maize Seed Endosperm.

T2 or T3 seed from self-pollinated maize plants transformed with either pNOV 4080 were obtained. The pNOV 4080 construct targets the expression of the R1 in the endosperm. Normal accumulation of the starch in the kernels was observed, as determined by staining for starch with an iodine solution. The expression of R1 was detected by Western blot analysis using an antibody raised against a R1-peptide fragment (YTPEKEKEEYEAARTELQEEIARGA). The increased dikinase activity of R1 [Ritte G., Lloyd J. R, Eckermann N., Rottmann A., Kossmann J., Steup M., 2002, PNAS, 99(10) 7166-7171; Ritte G., Steup M., Kossmann J., Lloyd J. R., 2003, Planta 216, 798-801.] can also be detected in the extract made from the endosperm of the transgenic corn overexpressing R1-protein.

### Example 3

Phosphorylated Starch from the Transgenic R1-corn.

Isolation of Starch from Corn:

The endosperm was obtained after removing the embryo and pericarp from the kernel, and kept on ice. To 12.6 g of endosperm add 60 ml of buffer (1.25 mM DTT, 10 mM EDTA, 10% glycerol, and 50 mM Tris-HCl, pH 7.0) and the mixture was homogenized. The homogenate was filtered through a layer of Miracloth (Calbiochem) to remove cell debris. Centrifugation of the filtrate was carried out at 15,000 g for 15 minutes, at 4° C. The delicate yellow gel-like layer on top of the packed white layer of sedimented starch granules was removed by gentle aspiration to obtaine clean-white granules. The resultant starch granules was washed twice with the buffer, twice with 80% ethanol to remove low molecular storage proteins, twice with cold acetone, and dried. The starch was isolated and stored at room temperature. [Chen Mu-Forster, Chee Harn, Yuan-Tih ko, George W. Singletary, Peter L. Keeling and Bruce P. Wasserman (1994) The Plant Journal 6(2), 151-159.]

Preparation of Starch Hydrolysate by Mild-Acid Hydrolysis of the Starch Sample:

Starch (100-500 mg) was suspended in 0.5-2.5 ml of 0.7 N HCl and kept at 95° C. for 4 hours. The glucose in the starch hydrolysate was quantified by glucose estimation kit (Sigma) and by HPLC analysis.

Glucose in the starch hydrolysate was oxidized to gluconic acid in the reaction catalyzed by Glucose Oxidase [from Starch/Glucose estimation kit (Sigma)]. The mixture was incubated at 37° C. for 30 minutes. Hydrogen peroxide released during the reaction changes the colorless o-Dianisidine to brown oxidized o-Dianisidine in presence of Peroxidase. Then, 12 N sulfuric acid was added to stop the reaction and to form a stable pink-colored product. Absorbance at 540 nm was measured for quantification of the amount of glucose in the sample, with respect to standard glucose solution.

An aliquot of the sample was diluted 5 to 25-fold, filtered through 0.2-micron filter for HPLC analysis.

The samples were analyzed by HPLC using the following conditions:

Column: Alltech Prevail Carbohydrate ES 5 micron 250×4.6 mm

Detector: Alltech ELSD 2000

Pump: Gilson 322

Injector: Gilson 215 injector/diluter

Solvents: HPLC grade Acetonitrile (Fisher Scientific) and Water (purified by Waters Millipore System)

Gradient used for oligosaccharides of low degree of polymerization (DP 1-15).

| Time | % Water | % Acetonitrile |
|---|---|---|
| 0 | 15 | 85 |
| 5 | 15 | 85 |
| 25 | 50 | 50 |
| 35 | 50 | 50 |
| 36 | 80 | 20 |
| 55 | 80 | 20 |
| 56 | 15 | 85 |
| 76 | 15 | 85 |

Gradient used for saccharides of high degree of polymerization (DP 20-100 and above).

| Time | % Water | % Acetonitrile |
|---|---|---|
| 0 | 35 | 65 |
| 60 | 85 | 15 |
| 70 | 85 | 15 |
| 85 | 35 | 65 |
| 100 | 35 | 65 |

System used for data analysis: Gilson Unipoint Software System Version 3.2 Estimation of sample sugar was done by integration of the peak-area generated on a HPLC profile and comparison with calibration-curve (peak-area vs weight) obtained using authentic sugar standards.
Glucose 6-Phosphate Dehydrogenase Assay to Determine the Level of Phosphorylation at the 6-Position of Glucose Residues in Starch:

To an aliquot (100 μl) of the mild-acid starch hydrolysate sample 800 μl of buffer containing 100 mM MOPS-KOH (pH 75), 100 mM MgCl₂, 2 mM EDTA in a cuvette and neutralize with 80-100 μl of 0.7 N KOH. The reaction was started by adding NAD (final concentration 0.4 mM) and 2 unit of Glucose 6-Phosphate dehydrogenase in a final assay volume of 1 mL. The reaction rate was calculated by measuring the change in absorption at 340 nm for 2 minutes.

[Nielsen, T. H., Wichmann, B., Enevoldsen, K., and Moller, B. L. Plant Physiol. (1994) 105, 111-117.]

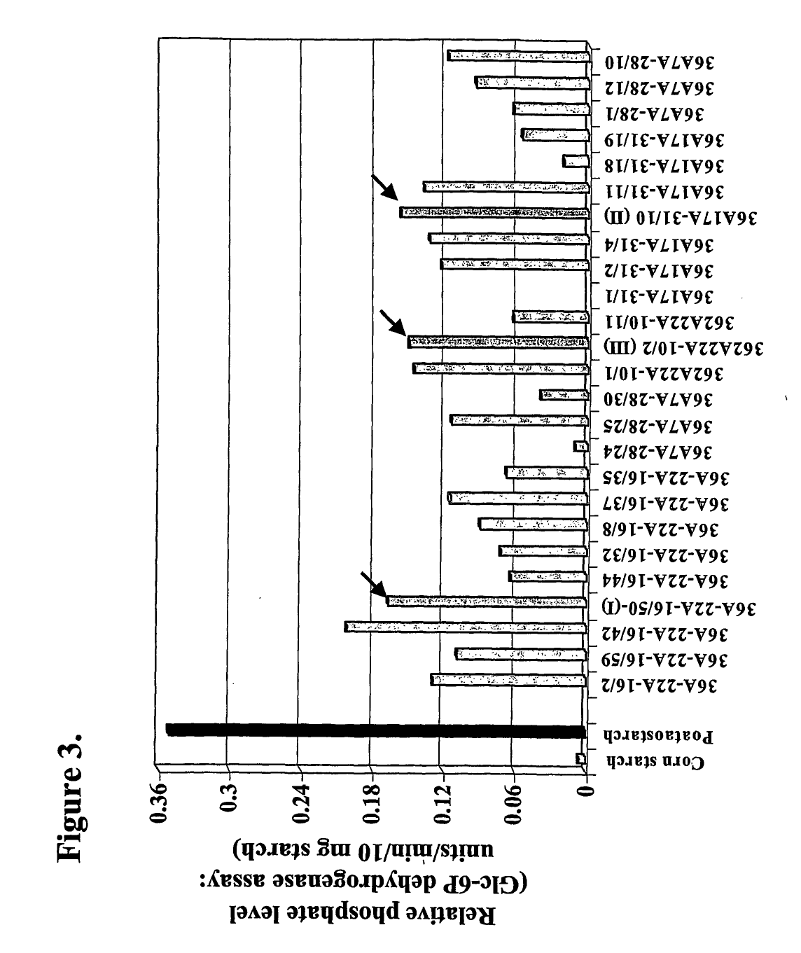

FIG. 3. Estimation of glucose 6-phosphate after complete hydrolysis of starch. Increased phosphorylation of R1-cornstarch. Starch samples (˜100 mg) isolated from the corn kernels (T3 seeds) of different events (transgenic R1-corn) were completely hydrolyzed (mild-acid hydrolysis, as described above) to glucose. The glucose and glucose 6-phosohate in the hydrolysates were quantified as described above. FIG. 3 shows the relative level of phosphorylation of the starch in different samples, as measure by the glucose 6-phospahte dehydrogenase assays and normalized with respect to the estimated glucose in the samples.

Screening of different R1-transgenic corn events using method above described method indicated high level of in planta phosphorylation of starch in corn expressing potato R1-transgene. The starch sample isolated from non-transgenic corn is not phosphorylated, as it is hardly detectable by this assay. The level of phosphorylation that is observed in case of R1-cornstarch is almost half the level that is observed in potato starch (commercially available sample). It is to be noted that this assay method detects the phosphorylation at the 6-position only, phosphorylation at any 3- or 2-position of glucose residue of starch is not detectable by this method. The three events (labeled as I, II & III, indicated with arrow) were used for further characterization (experiments described below) of the R1-corn.

³¹P-NMR Analysis to Estimate the Level of Ester-Linked Phosphate in Starch Sample:

Mild-acid hydrolyzed starch sample was cooled down to room temperature, buffered with 100 mM acetate buffer (pH 5.5) and finally neutralized with 2.8 N KOH. The samples were blown down under a stream of N₂ gas. A known amount of β-NAD was added to the sample. The sample was dissolved in 300 μl H₂O and 300 μl DMSO d₆. Spectral data was acquired on a DPX-300 at 30° C. β-NAD was used as the standard, used for quantification of the ester-linked phosphate in the sample. The quantification was carried out by intregation of the peak. Estimation of the phosphate level in the sample took into account the any presence of contaminating inorganic phosphate in the sample.

TABLE 1
Estimation of covalently-bound phosphate by 31P-NMR. The % phosphate
shown here is amount of ester-linked phosphate present in the starch
hydrolysate compared to the glucose in the sample. The experiments were
carried out as described above.

| STARCH SAMPLE | PHOSPHATE (%) |
|---|---|
| Non-transgenic cornstarch | 0 |
| R1-Cornstarch I (T3 seeds) | 0.0736 |
| R1-Cornstarch II (T3 seeds) | 0.0634 |
| R1-Cornstarch II (T3 seeds) | 0.0388 |
| Potato Starch | 0.1263 |

This result corroborates with that obtained by the glucose 6-phosphate assay; the phosphorylation level observed in R1-cornstarch samples was up to half of that observed in case of potato starch. Unlike, the glucose 6-phosphate assay method described above, this method estimates the total ester-linked phosphate associated with the starch sample.

### Example 4

Swelling and Solubility of R1-cornstarch.

The starch samples from the R1-corns, non-transgenic corn and the transgenic negative-control corn were prepared as described above; while the other starch samples were commercially obtained. The swelling power of starch samples were determined as described by Subramanian et al. (Subramanian, V., Hosney, R. C., Bramel-Cox, P. 1994, Cereal Chem. 71, 2772-275.) with minor modifications. The 1% (w/w) suspension of starch and distilled water was heated to 95° C. for 30 minutes. Lump formation was prevented by shaking. The mixture was centrifuged at 3000 rpm for 15 minutes. The supernatant was carefully removed and the swollen starch sediment was weighed the swelling power was the ratio in weight of the wet sediment to the initial weight of the dry starch.

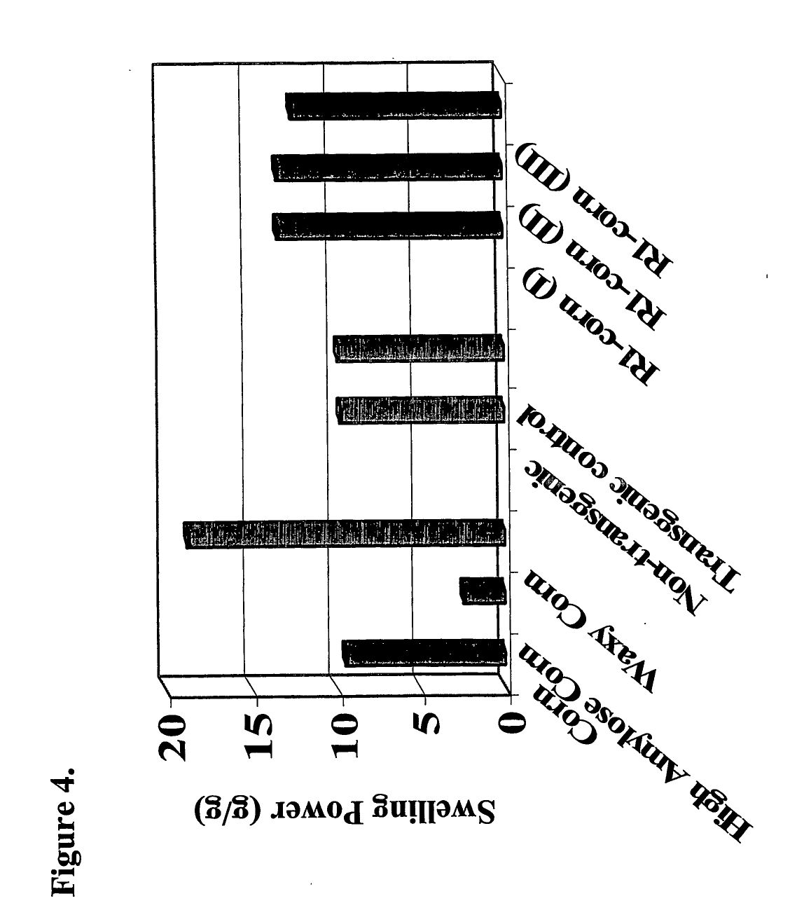

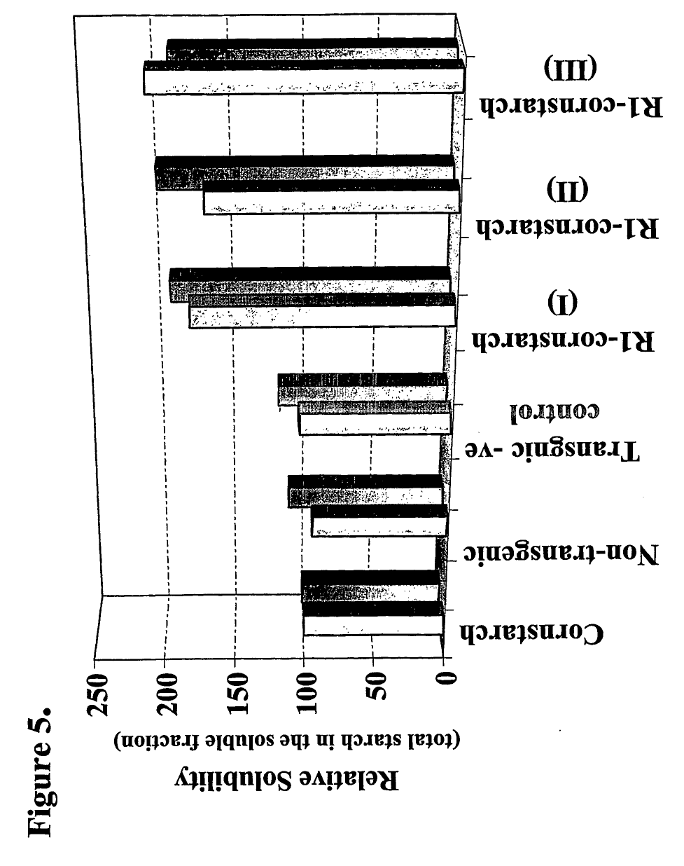

FIG. 4 shows the relative swelling-power of R1-cornstarch compared to non-transgenic cornstarch. The solubility of the starch samples were compared as follows. Starch sample (1% w/w) in 4.5 M urea was stirred for 30 minutes at 50° C. The mixture was centrifuged at 3000 rpm for 15 minutes. The supernatant was carefully removed. The starch present in the supernatant was estimated by Starch/Glucose estimation kit (Sigma) and by iodine staining. FIG. 5 shows the relative solubility of the R1-cornstarch compared to the non-transgenic cornstarch. Results from two independent set of experiment shown in the figure.

FIG. 5 shows the relative solubility of R1-cornstarch compared to non-transgenic cornstarch.

Phosphate, as a doubly-charged functional group, has high affinity for water; also, when covalently-bound to the glucose strands of starch the phosphate groups can assist swelling through electrical repulsion. Thus, by phosphorylating cornstarch its solubility in ionic solvents (including water) and its swelling power (e.g. in water) can be increased R1-cornstarch is a phosphorylated form of cornstarch, which usually is not phosphorylated. Hence, as expected, we observe increase in the swelling power (by 30-40%, FIG. 4) of R1-cornstarch (from T2 seeds of corn expressing potato R1 gene). The relative solubility (FIG. 5) of R1-cornstarch (from T2 seeds) also appears to be significantly higher than observed in case of non-transgenic control.

Susceptibility of R1-corn to Enzymatic Hydrolysis.

Susceptibility to Hydrolysis Under Simulated Digestive Conditions:

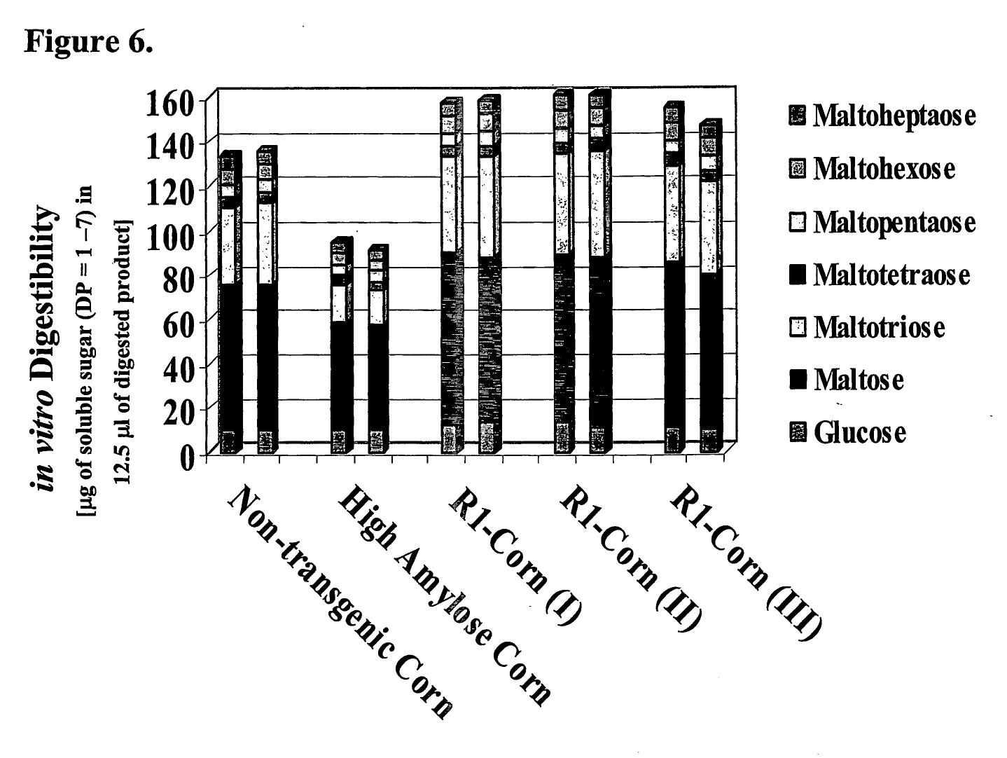

The samples for the assay were prepared by passing ground-up corn flour (seeds grinded in Kleco) through a sieve having a 300 micron pore-size. The sample (500 mg) was treated, for 30 min at 37° C. (on a reciprocating shaker), with 5 ml of pepsin/HCl (2000 units/ml in 0.1 N HCl) solution in acidic pH, simulating gastric digestive conditions. The incubated reaction mixture was then neutralized with NaOH and the next step of digestion was carried out with 2.5 ml pancreatin (5 mg/m in 150 mM KPO4, pH 7.0 buffer). The tube was vortexed and incubated with shaking on the reciprocating shaker at low speed at 37° C. for 120 min. At the end of the incubation 7.5 ml of water was added to each tube and vortexed. The undigested portion of the corn flour was precipitated by centrifugation in a table-top centrifuge at 24° C., 400 rpm for 30 min and the supernatant of the sample was heated at 100° C. for 15 miutes, allowed to cool, centrifuged and the supernatant was used to assay the amount of the total soluble sugar (measure glucose with BCA reagent after complete enzymatic hydrolysis of the sugar chain), small oligosaccharides (HPLC analysis described above) and glucose (BCA reagent) released due to digestion. The results obtained from different assay methods corroborated with each other. Shown in FIG. 6 is the HPLC analysis; the results clearly demonstrate an increase (10-20%) in the release of total small oligosaccharides (degree of polymerization 1-7) from R1-corn flour samples, as compared to the normal corn flour.

FIG. 6 demonstrates in vitro digestibility of R1 -corn flour under simulated digestive conditions. The figure shows the pile-up of the glucose and other small (<8) oligosaccharides obtained at the end of the simulated GI-track digestion process. The sugars are estimated by integration of the peak-area in the HPLC analysis profile.

Susceptibility to Hydrolysis in Presence (in vitro) of Different Starch Hydrolyzing Enzymes:

Enzymatic digestibility of R1-cornstarch in R1-corn flour was tested using three α-amlylases from different sources and one glucoamylase. The corn flour samples for the assay were prepared by passing ground-up corn flour (seeds grinded in Kleco) through a sieve having a 300 micron pore-size. Corn flour (50 mg) suspended in 500 μl of 100 mM sodium acetate (pH 5.5) was used for each enzyme reaction. In all these enzymatic digestions the amount of enzyme used was below the level required for complete hydrolysis of the available starch in the sample. The reactions were carried out with or without pre-incubation in the absence of enzyme as indicated in the figure legends.

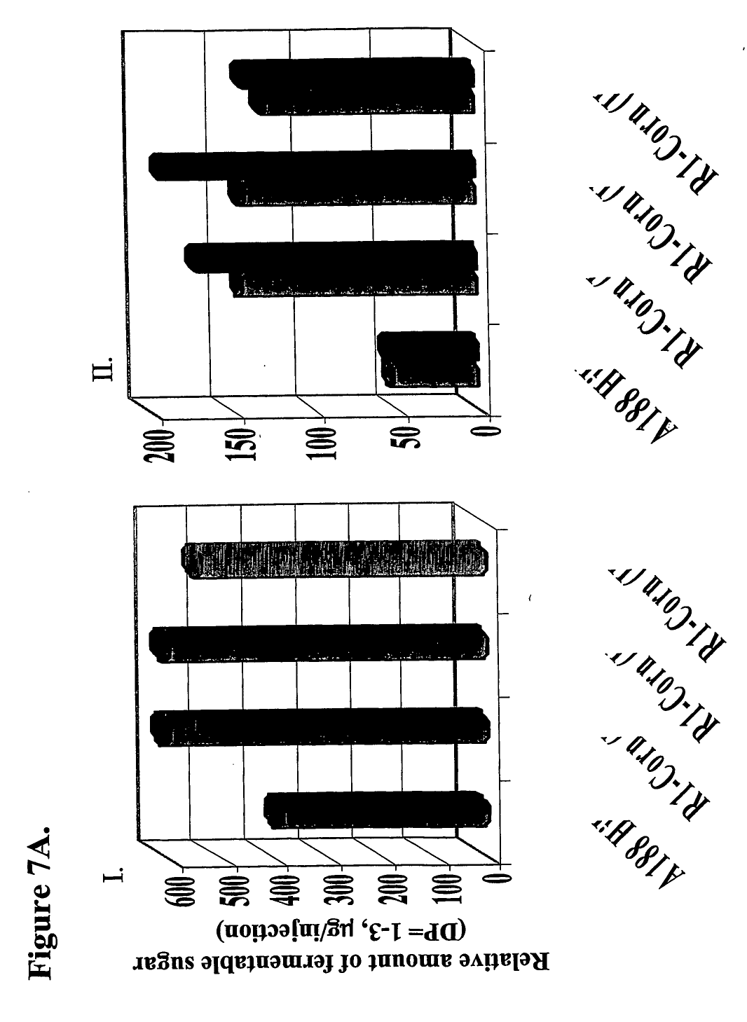

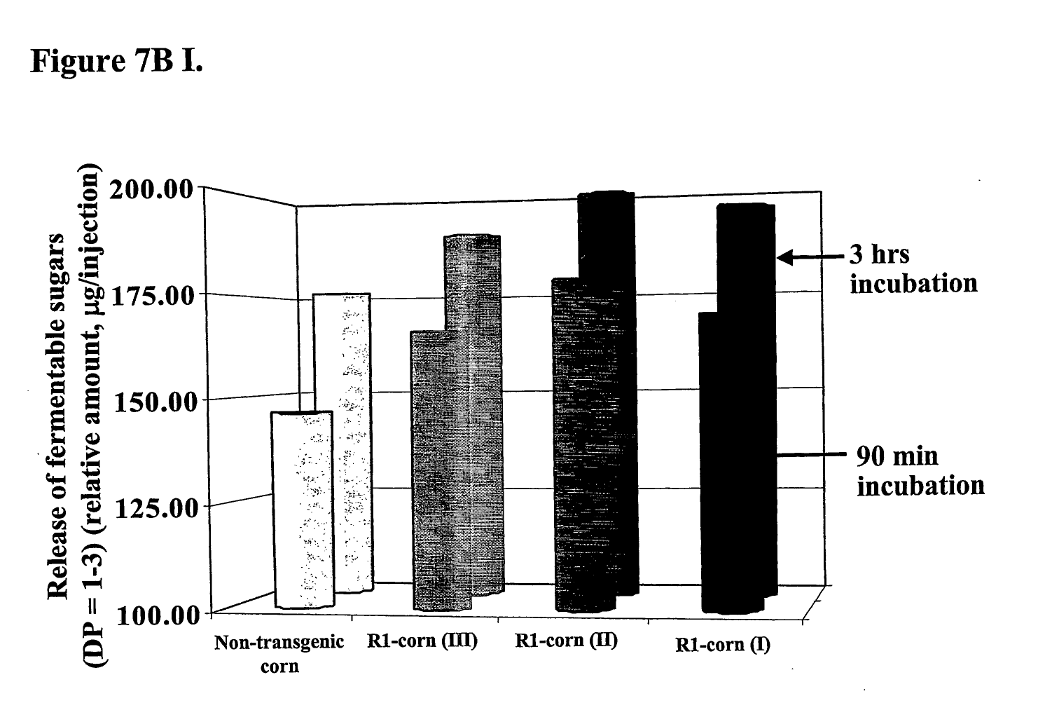

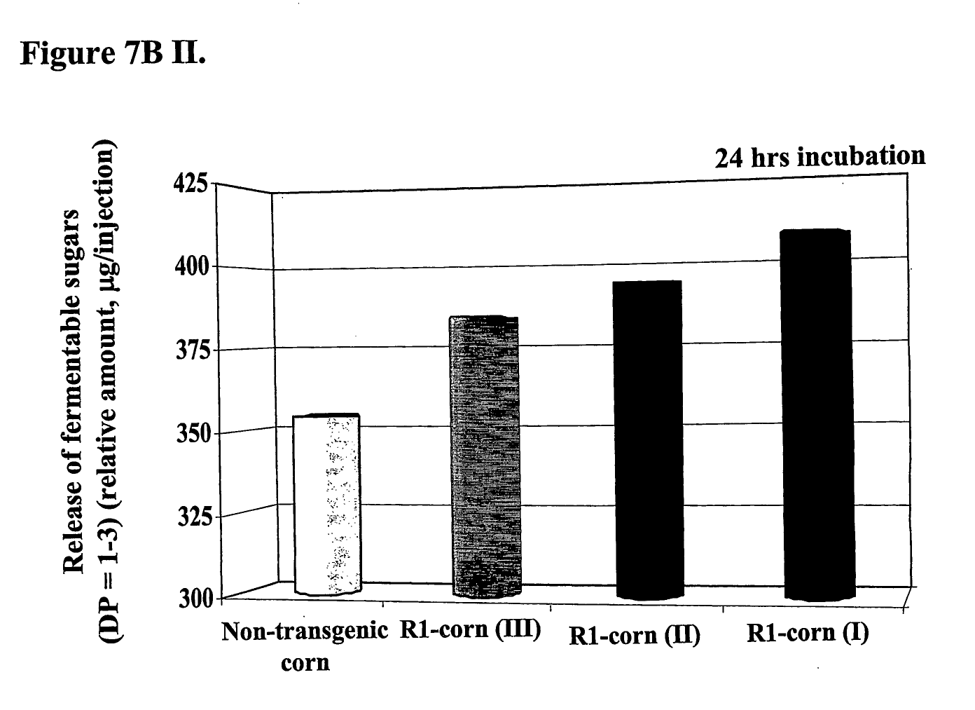

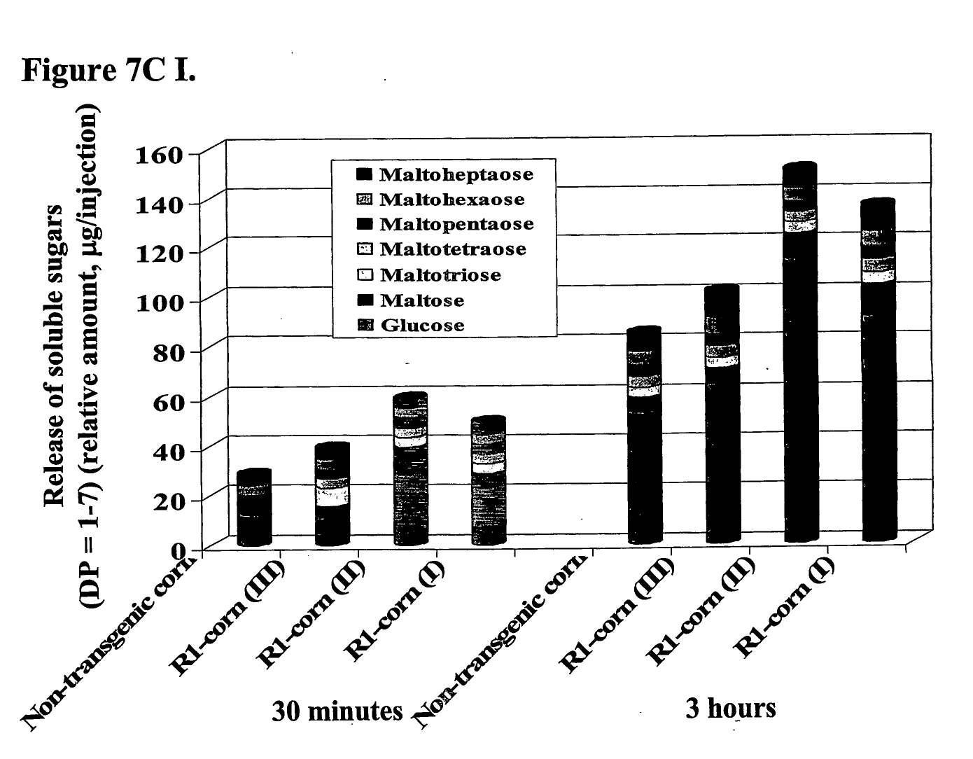

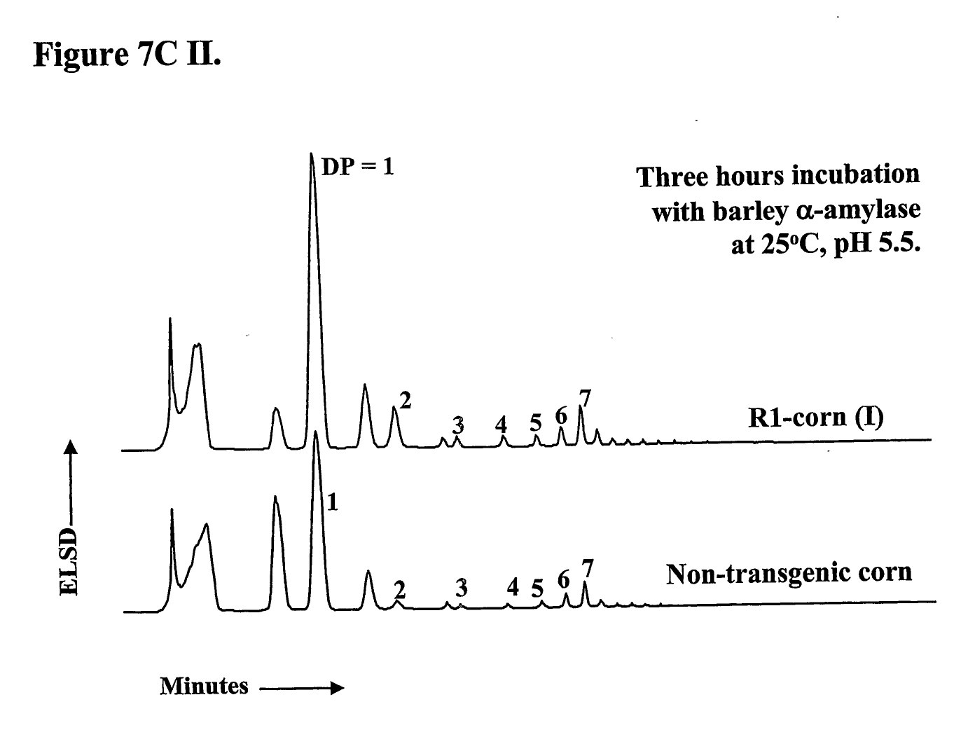

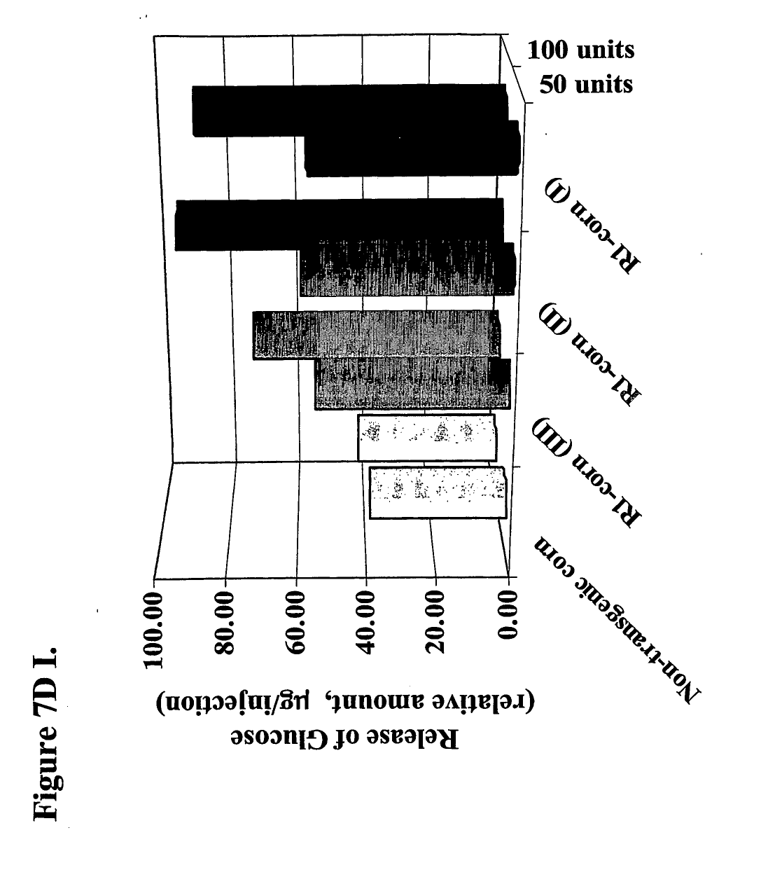

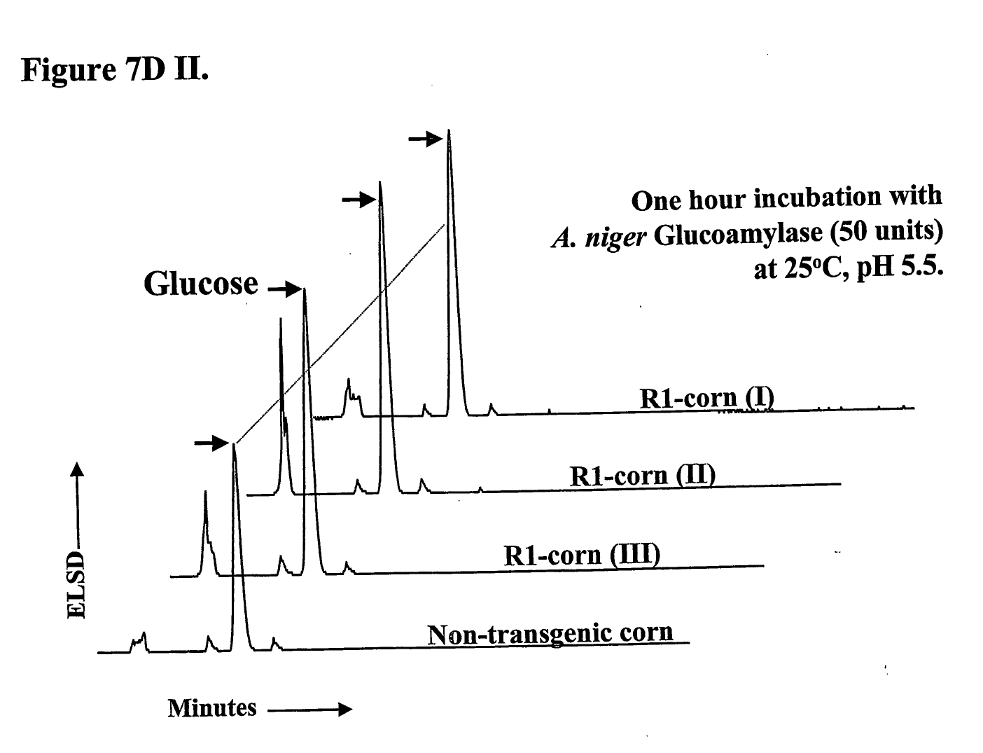

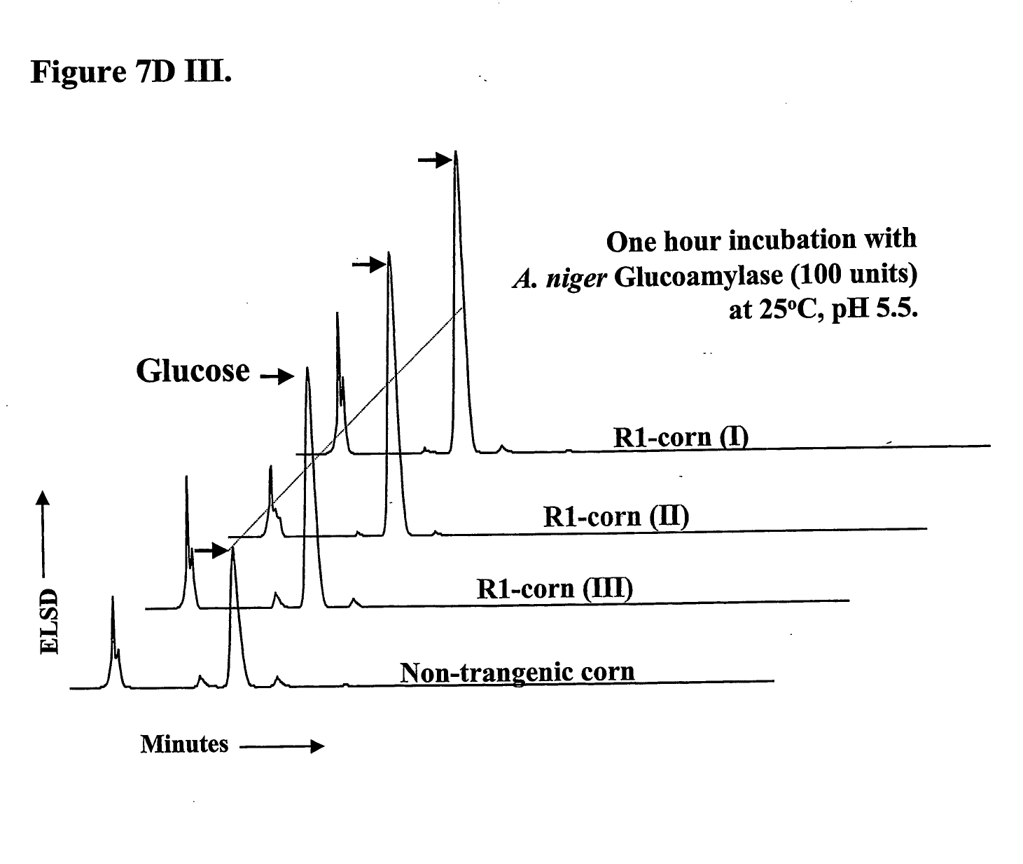

FIG. 7 shows the susceptibility of R1-corn flour to enzymatic hydrolysis by starch hydrolyzing enzymes. For results depicted in FIG. 7A, corn flour sample in sodium acetate buffer was pre-incubated at 75° C. (I) at 60° C. or 25° C. (II) for 15 minutes. At the end of the pre-incubation, the samples were cooled down to room temperature, 10 μl of α-amylase from Aspergillus oryzae (Sigma) was added each reaction mixture, vortexted and the incubation for 30 minutes at room temperature was carried out with constant shaking. The reaction mixture was then centrifuged at 14000 rpm for 2 minutes, the supernatant was collected and heated at 95° C. to deactivate any residual enzyme, centrifuged and the supernatant was filtered through 0.4 micron filter to prepare sample for HPLC analysis (method described above). The figure depicts the relative amount of easily soluble fermentable glucose oligosaccharides (Degree of polymerization=1-3) released as a result of the enzymatic hydrolysis. The amount of fermentable sugars is the sum of amount of glucose, maltose and maltotriose product, estimated from the HPLC analysis (integration of peak area and comparison with calibration-curves generated with authentic sugars).

The difference in the relative susceptibility to hydrolysis is much more prominent when the corn-flour samples were not heated above the gelling-temperature (˜70° C., during pre-incubation or incubation with enzyme) of cornstarch (FIG. 7).

For FIG. 7B, susceptibility of different corn flour samples towards a thermophillic α-amylase (expressed as transgene in corn) was carried out in similar manner as describe in case of A. oryzea α-amylase. Corn flour sample (non-transgenic control and R1-corn) was mixed with the flour from corn expressing the α-amylase in 10:1 ratio and incubated at 85° C., for 90 minutes (I), 3 hours (I) or 24 hours (II). The released soluble sugar analyzed and quantified by HPLC, as described previously.

For FIG. 7C, digestibility of non-transgenic corn and R1-corn samples (50 mg) towards α-amylase from barley (10 μl of purified enzyme, protein concentration 5 mg/ml) was measured by mixing enzyme after 15 minutes pre-incubation at room temperature. The reaction was carried out as described in case A. oryzea α-amylase. Incubation at room temperature was done for 30 minutes and 3 hours. The FIG. 7C I, shows the relative amount of soluble glucose oligosaccharides released after the enzymatic reaction; while the HPLC profiles generated for one of the R1-corn sample and the non-transgenic corn sample are shown in FIG. 7C II.

FIG. 7D shows the results of an experiment similar to those described above was also carried out with Glucoamylase from Aspergillus niger (Sigma) as the enzyme and non-transgenic corn or R1-corn sample (50 mg) as the substrate. Enzyme (50 or 100 units) was mixed with corn flour sample (in 100 mM sodium acetate buffer pH 5.5) that is pre-incubated at room temperature and the incubation was continued at room temperature for 60 minutes. The glucose released into the reaction mixture was analyzed by HPLC as described above. The FIG. 7D I, shows the relative amount of glucose produced after the enzymatic reaction; while the HPLC profile generated for the R1-corn and the non-transgenic corn samples are shown in FIG. 7D III (100 units of enzyme).

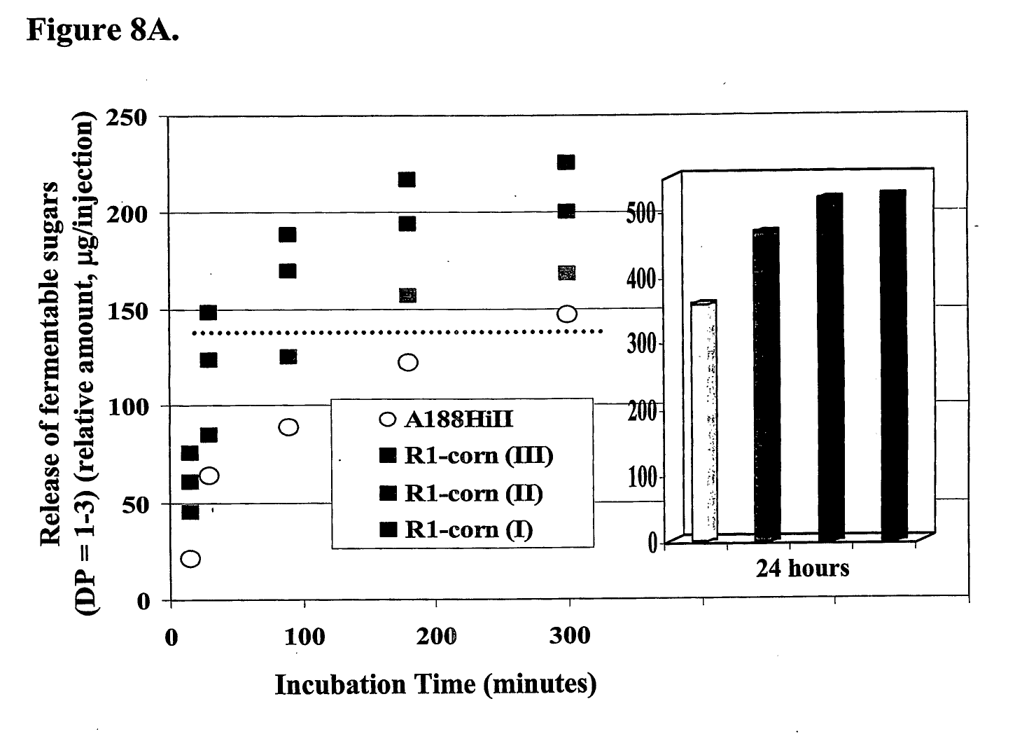

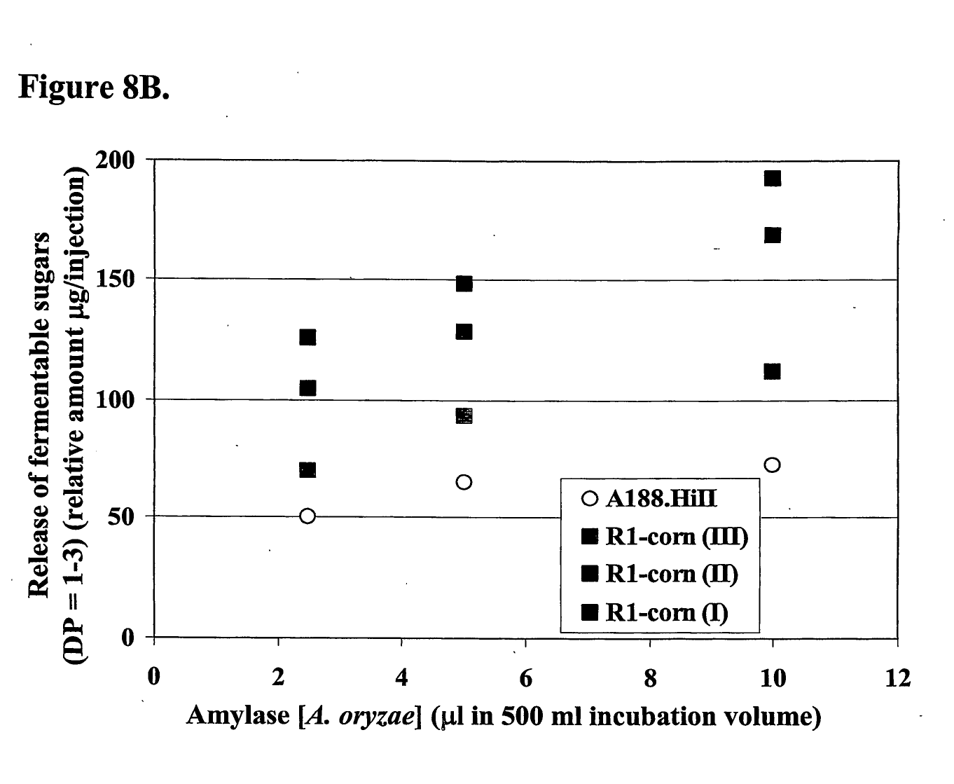

FIG. 8 shows the effect of incubation time and enzyme concentration on the rate of hydrolysis of R1-cornstarch.

The experiment was carried out as described previously in case of FIG. 7A. The pre-incubation and incubation temperature is 25° C. (room temperature). The amount of enzyme [α-amylase (A. oryzae)] used to test the effect of incubation time on the hydrolysis is 10 μl in 500 μl of reaction volume (FIG. 8A). Incubation time for the experiment shown in FIG. 8B is 30 minutes. As shown above, covalent derivatization of starch with hydrophillic functional group(s) (e.g. phosphate, as in case of R1-cornstarch) increases its swelling as well as solubility in aqueous medium, making the modified starch molecules more accessible to hydrolytic enzymes. Hence, the use of such a derivatized form of starch not only will increase the proportion of starch available for enzymatic degradation it possibly can also increase the rate of hydrolysis. Here, this hypothesis is tested using phosphorylated cornstarch that was made in transgenic corn plant by expressing R1-protein gene from potato in the endosperm of corn.

As shown in FIG. 6, the R1-cornstarch (in corn flour) is comparatively more digestible (as measured by the in vitro assay) compared to normal cornstarch (non-transgenic). In this in vitro assay effort has been made to mimic the enzymatic reaction conditions found in the digestive track of mono-gastric animal. A difference of more that 10-15% was found between the R1-corns samples and the control non-transgenic corn.

Compared to cornstarch, R1-cornstarch is also more susceptible to attack by all the starch hydrolyzing enzymes tested (FIGS. 7 & 8). This again is consistent with the idea that R1-starch, being phosphorylated, swells and hydrates more (compared to normal cornstarch, which is not phosphorylated) in aqueous solution, making the R1-starch molecules more accessible to attack by hydrolytic enzymes. Collectively the experiments described in FIGS. 7 & 8 demonstrate that the R1-cornstarch in corn flour is hydrolyzed at a faster rate compared to non-transgenic control. Thus, same amount of fermentable/soluble glucose oligosaccharides can be released from R1-cornstarch by using less amount of enzyme and/or with shorter period of incubation that that is required for non-transgenic control starch.

It should be also noted that the difference in the relative susceptibility to hydrolysis was more prominent when the corn-flour samples were not heated above the gelling-temperature (˜70° C., during pre-incubation or incubation with enzyme) of cornstarch (FIG. 7).

### Example 5

Fermentability of R1-corn Starch.

Fermentation procedure: Corn flour sample of the transgenic and non-transgenic corn were prepared by grinding corn kernel to a fine powder (>75% of the weight passes a 0.5 mm screen) using a hammer mill (Perten 3100). The moisture content of the corn flour samples were determined using a Halogen Moisture Analyzer (Metler). Typically the moisture content of the samples ranges between 11-14% (w/w). Corn flour samples were weighed into 17×100 mm polypropylene sterile disposable culture tubes. The approximate weight of the dry sample is 1.5 g per tube. In each tube 4 ml water was added and the pH is adjusted 5.0. Each samples were inoculated with ˜1×10⁷ yeast/g flour. [The yeast (EDT Ferminol Super HA—Distillers Active Dry Yeast) inoculum culture was grown in Yeast starter medium (300 ml containing 50 g M040 maltodextrin, 1.5 g Yeast extract, 0.2 mg ZnSO₄, 100 μl AMG300 glucoamylase and ml of tetracycline (10 mg/ml)). The medium was inoculated with 500 mg yeast and incubated at at 30° C. for 16 h, with constant shaking.] The inoculation was followed by addition of 0.5 ml of yeast extract (5%), 1.5 ml water, 0.03 ml 0.9 M sulphuric acid and Glucoamyalse (Aspergillus niger) Sigma A7095-50ML. The final fermentation mixture is adjusted to 33% solid. The fermentation tubes were weighed and incubated at 30° C. The tubes were weighed, without mixing, at intervals (at least once/24 h) weigh the tubes. Aliquot of samples were also taken out from the fermentation tubes (after mixing) at regular interval (every 24 hours) for estimation of ethanol production HPLC analysis (described below).

HPLC-analysis of the fermentation products. This method is used to quantify the ethanol and other fermentation products produced during the corn fermentation process. Waters 2695 Alliance HPLC System equipped with binary pump and temperature controlled auto sampler; Waters 2414 Refractive Index Detector and a Column Heater from Eppendorf were used fro the analysis.

Chromatography Conditions:
Column Type: Bio-Rad Aminex HPX-87H (300×7.8 mm)
Column Temperature: 50 C
Detector Temperature: 35 C
Sample Temperature: 6-11 C
Mobile Phase: 0.005 M Sulphuric Acid in HPLC grade water
Flow rate: 0.6 mL/min
Isocratic
Run Time: 30 minutes
   A 5-point calibration curve is generated and used to quantitate ethanol and other fermentation products. For calibration the various compounds (Maltodextrin M 100 (DP 4+), Maltotriose (DP 3), Maltose, Glucose, Fructose, Lactic Acid, Glycerol, Acetic Acid and Ethanol are weighed or pipetted into a 100 mL volumetric flask and diluted to volume with 0.02% Azide in HPLC grade Water. Standards: A 25 υL of Std-0%; Std-5%; Std-10%; Std-15% and Std-20% are injected to make the 5-point calibration curve. Std-0 is the blank. Sample: A 25 υL sample fermentation mixture (after centrifugation at 14,000 rpm for 5 minutes and filtering through 0.2 micron filter) is injected.

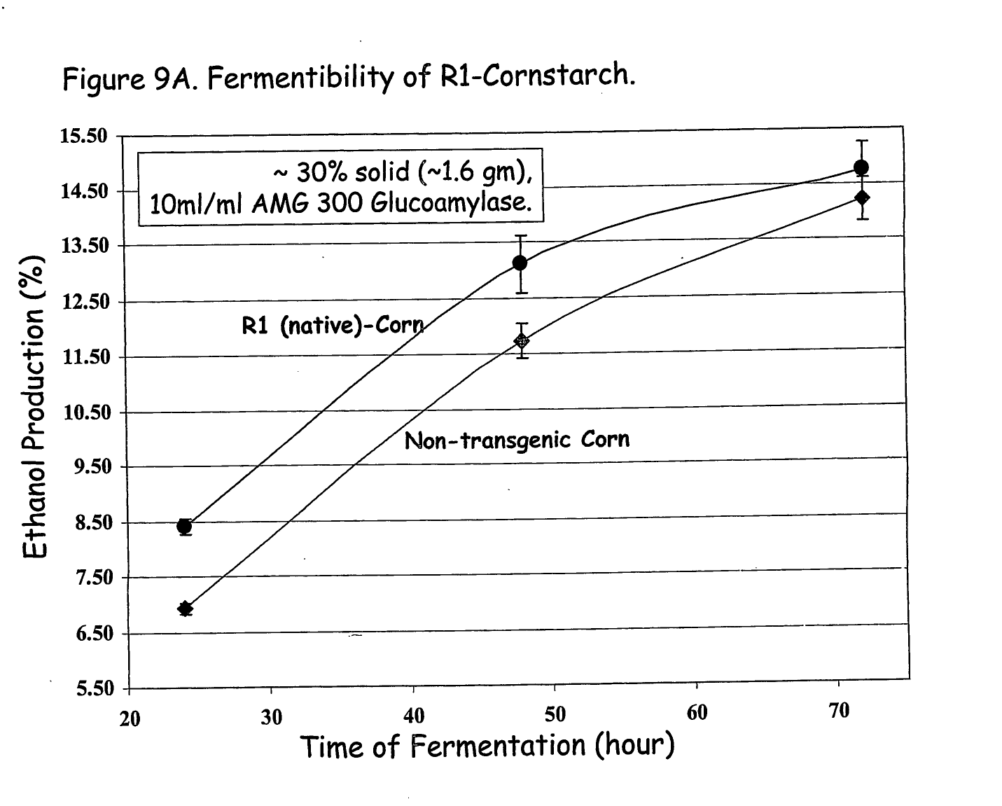

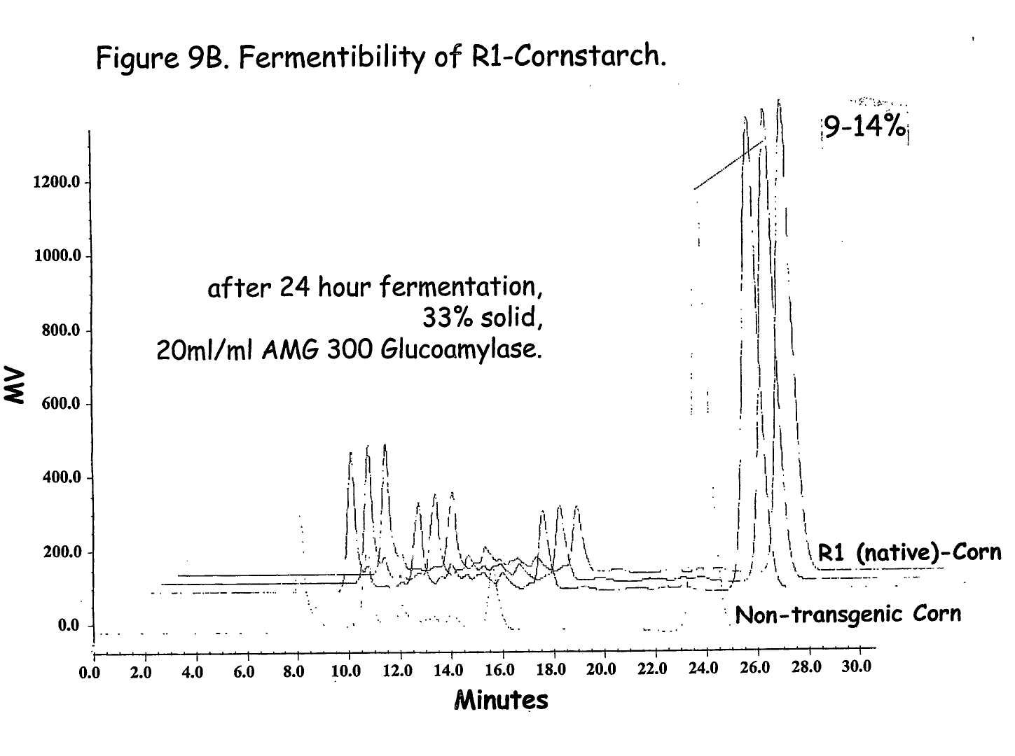

FIGS. 9A & 9B show the results obtained with samples of transgenic corn expressing potato native R1-gene; these results being compared to the non-transgenic control. We found that the transgenic samples performed better (˜9-14% at 24 hours) in the fermentation process with regard to the ethanol production; this trend continued for at least 72 hours of fermentation, although the trend appeared to decrease with the progress of the time of incubation. Consistent with this observation we also find that the percent weight change per unit dry weight also higher (1-3%) in case of transgenic R1-corn, compared to the control.

This find is consistent with the our hypothesis that the phosphorylated form of corn starch due to its higher swelling power and solubility in water can easily targeted by hydrolytic enzymes. This will lead to efficient hydrolysis of the phosphorylated starch, at a faster rate and/or using lesser amount of enzyme, compared to normal non-phosphorylated starch. The efficient hydrolysis and efficient release of fermentable sugar enables increase yield in ethanol production, as demonstrated here. This result may be extrapolated for other kinds of fermentation products (lactic acid, glycerol etc.).

### Example 6

Phosphorylated Starch from the Transgenic-corn Expressing Synthetic Version of Maize-codon Optimized Potato R1-gene.

The isolation procedure for starch from corn kernel, mild acid-hydrolysis of the isolated starch samples, glucose and glucose 6-phosphate estimation were carried out as described previously.

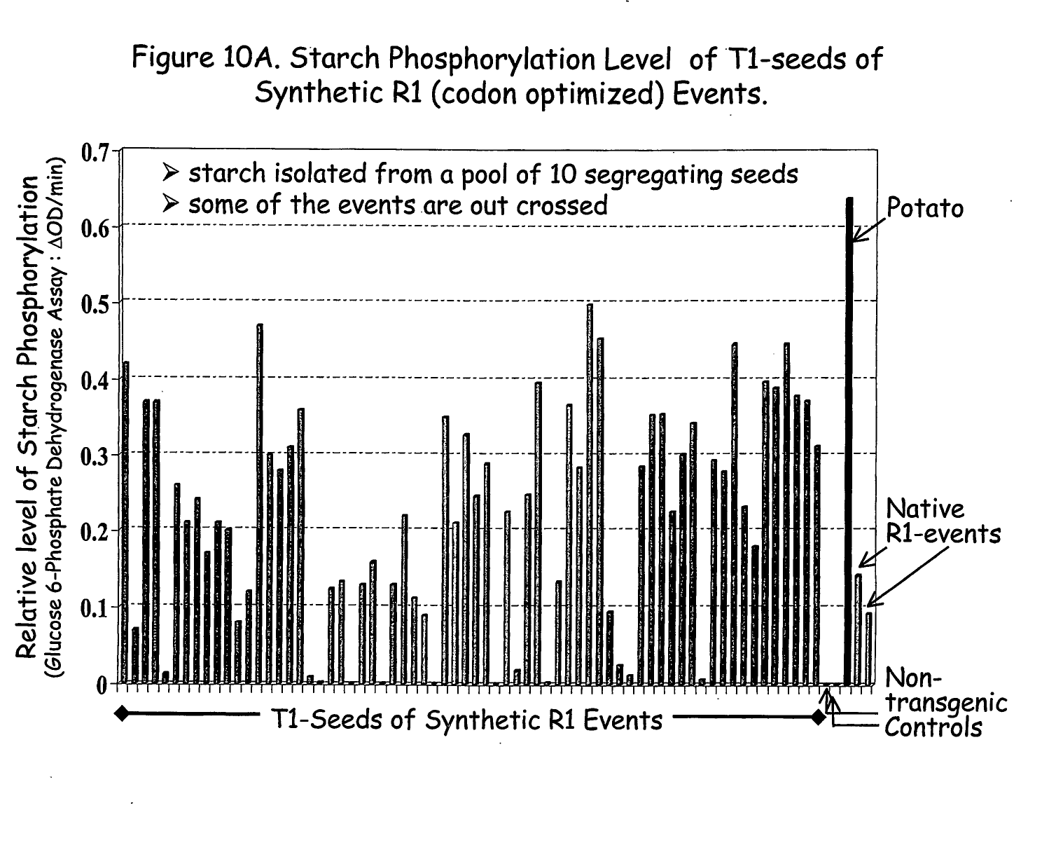

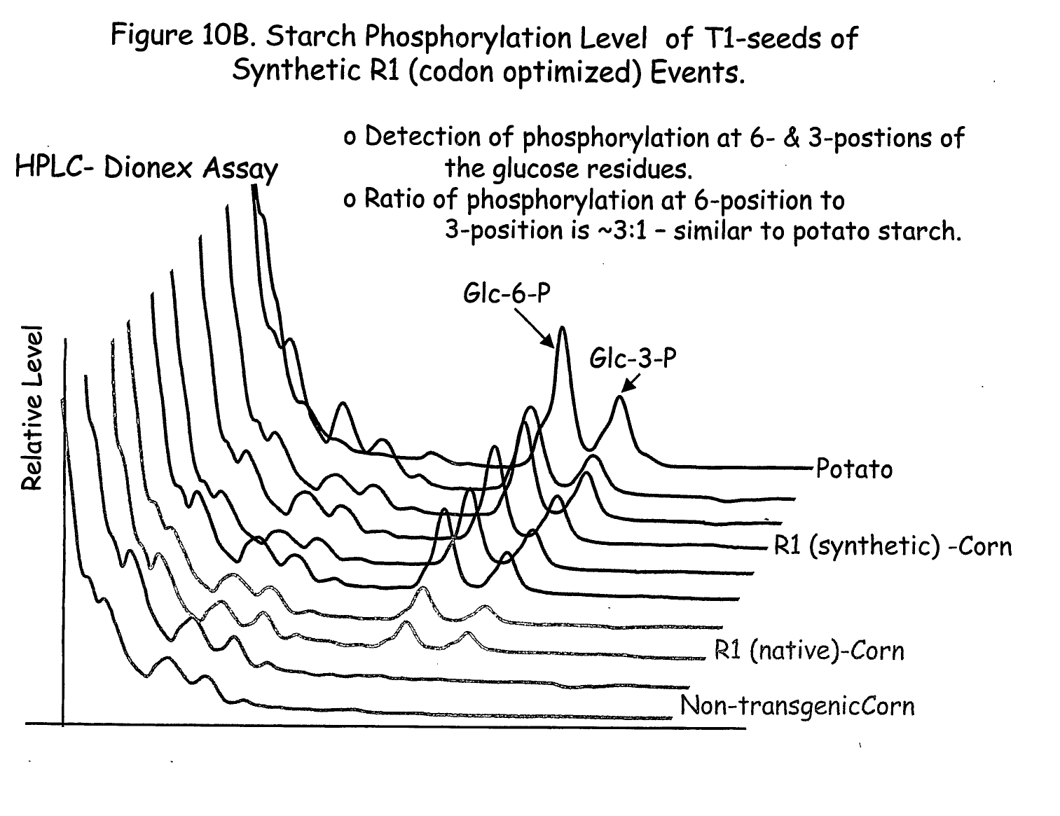

FIG. 10A provides an estimation of glucose 6-phosphate after complete hydrolysis of starch. Increased phosphorylation of R1(synthetic)-cornstarch. Starch samples (˜100 mg) isolated from the corn kernels (T1 seeds) of different events (transgenic synthetic R1-corn) were completely hydrolyzed (mild-acid hydrolysis, as described above) to glucose. The glucose and glucose 6-phosohate in the hydrolysates were quantified as described above. FIG. 10A shows the relative level of phosphorylation of the starch in different samples, as measure by the glucose 6-phospahte dehydrogenase assays and normalized with respect to the estimated glucose in the samples.

Screening of different synthetic R1-transgenic corn events using method above described method indicated high level of in planta phosphorylation of starch in corn expressing potato R1(synthetic)-transgene. The starch sample isolated from non-transgenic corn is not phosphorylated, as it is hardly detectable by this assay. The level of phosphorylation that is observed in case of maize-codon optimized synthetic R1-cornstarch considerably more than the level that is observed in transgenic corn expressing native potato R1 gene. It is to be noted that this assay particular method detects the phosphorylation at the 6-position only, phosphorylation at any 3-position of glucose residue of starch is not detectable by this method.

HPLC assay to quantify and detect Glucose 6-phosphate and Glucose 3-phosphate. In order to detect and quantify Glucose 6-phosphate and glucose 3-phosphate in the hydrolysate of the starch samples HPLC assays was carried out using Dionex DX-500 BioLC system consisting of: GS-50 Gradient Pump with degas option; ED 50 Electrochemical Detector; AS-50 Thermal Compartment; AS-50 Autosampler Chromatography conditions are:

1. Column Type:CarboPac PA 10 Analtyical (4×250 mm)

2. Detector Temperature: Ambient

3. Sample Temperature: Ambient

4. Eluents: A: Water B: 300 mM NaOH C: 1M NaOAC

5. Flow rate: 1.0 mL/min

6. Program:

| Time (min) | A (%) | B (%) | C (%) |
|---|---|---|---|
| 0 | 87.5 | 12.5 | 0.00 |
| 15.0 | 85.50 | 12.50 | 2.00 |
| 15.10 | 85.50 | 12.50 | 2.00 |
| 25 | 0.00 | 60.00 | 40.00 |
| 30.0 | 0.00 | 60.00 | 40.00 |
| 33.5 | 0.00 | 0.00 | 100.00 |
| 36.5 | 87.50 | 12.50 | 0.00 |
| 43.0 (End) | 87.50 | 12.50 | 0.00 |

7. Detection (ED4O): Pulsed amperometry, gold electrode. Waveform for
the ED4O:

| Time (s) | Potential (V) | Integration |
|---|---|---|
| 0.0 | 0.05 | |
| 0.20 | 0.05 | Begin |
| 0.40 | 0.05 | End |
| 0.41 | 0.75 | |
| 0.60 | 0.75 | |
| 0.61 | −0.15 | |
| 1.00 | −0.15 | |
| 0.20 | | |

D-Glucose-6-phosphate Dipotassium salt and Glucose 1-phosphate (Sigma) was used as the standards. A 5-point calibration curve is generated and used to quantify the level of glucose 6-phosphate.

FIG. 10B shows the elution profiles of some Dionex HPLC analysis of hydrolysates of starch samples from transgenic and non-transgenic corn and from potato. The second peak adjacent to the Glucose 6-phosphate peak is probably due to the presence of glucose 3-phosphate (this chromatohgarphy procedure was able to distinctly separate Glucose 6-phosphate and Glucose 1-phosphate) in the hydrolysates. A higher level of starch phosphorylation was observed in transgenic corn (segregating corn kernel from T1 seeds) expressing codon-optimized synthetic R1-gene compared to the starch samples isolated from transgenic corn expressing native potato R1-gene.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

We claim:

1. A method of producing modified maize starch comprising:
a) transforming a maize cell with an expression cassette containing a nucleic acid encoding R1;
b) producing said modified starch.

2. Modified maize starch produced by the method of claim 1.

3. Animal feed comprising the phosphorylated starch of claim 2.

4. A method of preparing fermentation product comprising:
a) preparing grain containing the phoshphorylated starch of claim 2;
b) adding the product resulting from a) to a fermentation reaction.

5. The method of claim 4 wherein the fermentation product is ethanol, lactic acid, acetic acid, or glycerol.

6. A method of improving starch digestibility in a monogastric animal comprising feeding grain comprising an expression cassette containing a nucleic acid encoding R1 to said animal.

7. A method of increasing the fermentable/hydrolyzable starch in grain comprising inserting an expression cassette containing a nucleic acid encoding R1 into said grain.

8. A method of using the modified starch of claim 2 in raw starch fermentation.

9. A method of preparing a solution of hydrolyzed starch product comprising treating a plant or plant part comprising starch granules under conditions which activate the R1 polypeptide thereby processing the starch granules to form an aqueous solution comprising hydrolyzed starch product.

10. The method of claim 9 further comprise isolating the hydrolyzed starch product and/or fermenting the hydrolyzed starch product.

11. A nucleic acid comprising SEQ ID NO: 1.
